(19) United States
(12) Patent Application Publication
(10) Pub. No.: US 2021/0153994 A1
(43) Pub. Date: May 27, 2021

(54) Dosing Indicator

(71) Applicant: The Procter & Gamble Company, Cincinnati, OH (US)

(72) Inventor: Samantha Chen-Yee Wang, Cincinnati, OH (US)

(21) Appl. No.: 17/100,977

(22) Filed: Nov. 23, 2020

Related U.S. Application Data
(60) Provisional application No. 62/940,933, filed on Nov. 27, 2019.

(51) Int. Cl. A61C 19/06; A61Q 11/00; A61K 8/22; A61K 8/21; A61K 8/27

(52) U.S. Cl. CPC A61C 19/066; A61Q 11/00; A61K 8/22; A61K 2800/872; A61K 8/27; A61K 2800/10; A61K 2800/92; A61K 8/21

(57) ABSTRACT

Devices with one or more dosing indicator symbols to signal to a user how much material should be dispensed. A whitening pen with a first dosing indicator on the body and a second dosing indicator on the rotating portion that can allow a user to dispense a known amount of a whitening composition.

### FIELD OF THE INVENTION

The present invention relates to a dosing indicator for indicating the dose supplied by a device. The present invention also relates to a whitening pen comprising a dosing indicator for indicating the amount of a whitening composition supplied from the whitening pen.

### BACKGROUND OF THE INVENTION

A variety of devices can be used to deliver whitening compositions directly to the teeth. Many whitening compositions, such as those composition comprising peroxide compounds, can lead to sensitivity amongst users. Unfortunately, many users of whitening compositions overdose whitening compositions, which can increase the sensitivity experienced.

Some dosing indicators for pharmaceutical compositions exist but are cost-prohibitive for use in consumer products, such as with whitening compositions. Accordingly, there is a need for a cost-effective dosing indicator for a device to deliver whitening compositions.

### SUMMARY OF THE INVENTION

Disclosed herein is a cost-effective dosing indicator for devices that dispense consumer products, such as whitening compositions and/or oral care compositions. The dosing indicator comprises one or more dosing indicator symbols, which can convert the distance rotated into amount of material dispensed.

Disclosed herein is a whitening pen comprising: (a) a whitening composition; (b) a body; (c) a rotating portion; and (d) a dosing indicator, the dosing indicator comprising: (i) a first dosing indicator symbol on the body, and (ii) a second dosing indicator symbol on the rotating portion.

Disclosed herein is a dispensing device comprising: (a) an oral care composition; (b) a body; (c) a rotating portion; (d) a dosing indicator, the dosing indicator comprising: (i) a first dosing indicator symbol on the body, and (ii) a second dosing indicator symbol on the rotating portion.

Disclosed herein is a dispensing device comprising: (a) an oral care composition, the oral care composition comprising fluoride or peroxide; (b) a body; (c) a rotating portion; (d) a dosing indicator, the dosing indicator comprising: (i) a first dosing indicator symbol on the body, and (ii) a second dosing indicator symbol on the rotating portion.

Disclosed herein is a method of dispensing an oral care composition from a dispensing device comprising: (a) providing a dispensing device, the dispensing device comprising: (i) the oral care composition comprising fluoride or peroxide; (ii) a body; (iii) a rotating portion; (iv) a dosing indicator comprising: (1) a first dosing indicator symbol on the body, and (2) a second dosing indicator symbol on the rotating portion; (b) aligning the first dosing indicator symbol with the second dosing indicator symbol; (c) rotating the rotating portion in one direction; and (d) re-aligning the first dosing indicator symbol with the second dosing indicator symbol.

### BRIEF DESCRIPTION OF THE DRAWINGS

FIG. 1 is a top view of a device including first and second dosing indicator symbols.

FIG. 2 is a cross-sectional view along a longitudinal axis of the device of FIG. 1.

FIG. 3 is a top view of a device that has been rotated by a user.

FIG. 4 is a cross-sectional view along a longitudinal axis of the device of FIG. 3.

### DETAILED DESCRIPTION OF THE INVENTION

The present invention is directed to a dosing mechanism to aid a user to dispense the correct dose of an oral care composition stored within a device. While dosing indicators have been disclosed for devices to dispense pharmaceutical compositions or for devices to be dispensed by a dental professional, there is a need for a cost-effective dosing indicator for use on devices to apply oral care compositions, such as whitening compositions.

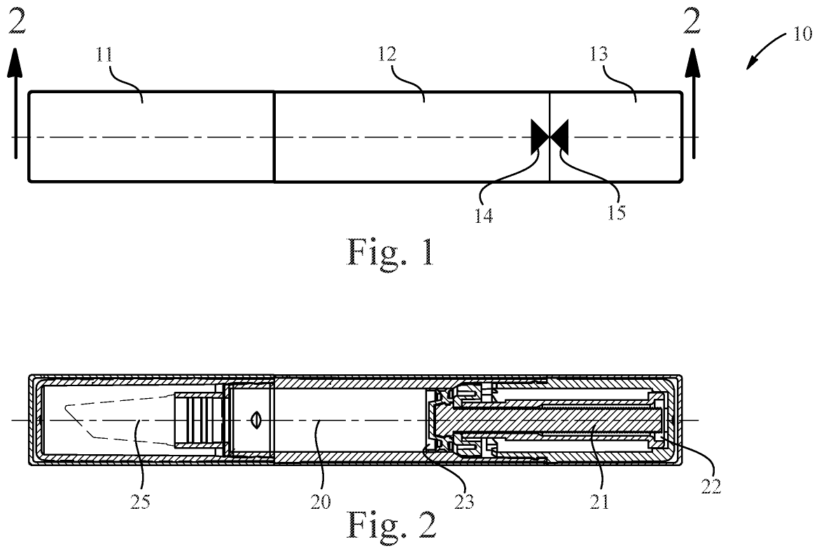

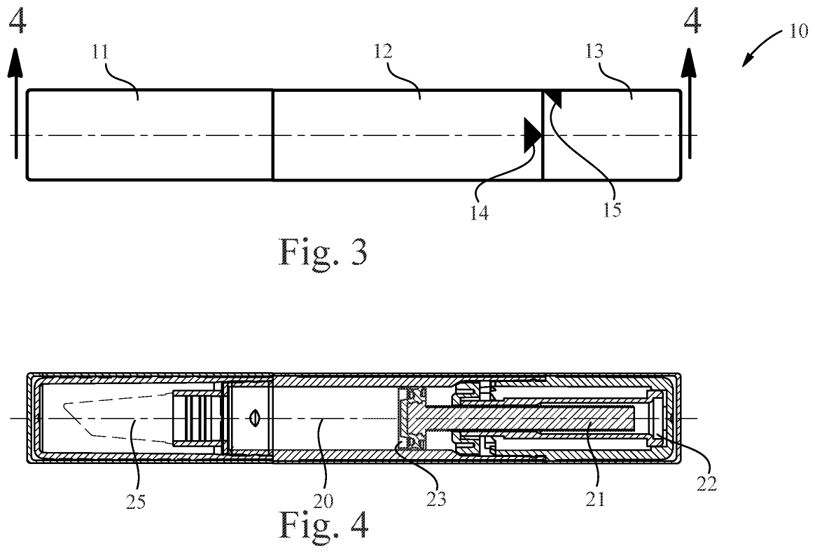

In twisting applicators, as exemplified in the pen-type twist device shown in FIG. 1 and FIG. 3, a user will manually rotate the rotating portion of the device. A rotation of the rotating portion of the device will move a platform at the bottom of a reservoir storing the oral care composition within the device towards an applicator tip, as shown in FIG. 3. The platform lowers the volume in the reservoir, which forces the oral care composition stored within the reservoir, out of one or more orifices in an applicator tip. In current twisting applicators, a user will rotate the rotating portion until a portion of the oral care composition has been expelled onto the applicator tip that can be applied to the oral cavity. Unfortunately, it is difficult to know how much material has been dispensed on the applicator tip beyond visual estimation.

The present invention is directed at a dosing indicator that can guide a user to dispense an exact amount of material. An exact dosing can be beneficial when dispensing oral care compositions comprising a whitening agent, fluoride, or other benefit agents that can require exact dosing to provide the benefit without leading to irritation of the oral cavity and/or extraneous dosing which could lead to accidental ingestion. The rotating portion and body (or stationary portion) can include a first and second dosing indicator symbol so that when the rotating portion is rotated, the user has a guide to how far in distance the rotating portion has been actually rotated. The amount rotated can correspond with the amount of oral care composition extruded onto the applicator tip. A full turn can be realized when the first and second dosing indicator symbols start aligned and the rotating portion is rotated a full turn to when the first and second dosing indicator symbols are aligned again, thereby indicating to a user to stop rotating the rotating portion. A full turn of the device can correspond to the correct dosage to apply to the oral care cavity. Additionally, a partial turn of the device can correspond to the correct dosage to apply to the oral cavity.

### Definitions

By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, tooth whitening composition, tooth coloring composition, or denture care or adhesive product.

“Elastomer,” as used herein, is defined as a polymer with rubber-like elasticity. An elastomer is a polymer with viscoelasticity, weak intermolecular forces, low Young's modulus, and high failure strain compared with other polymers and materials.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The foregoing summary is not intended to define every aspect of the invention, and additional aspects are described in other sections. In addition, the invention includes, as an additional aspect, all embodiments of the invention narrower in scope in any way than the variations defined by specific paragraphs set forth herein. For example, certain aspects of the invention that are described as a genus, and it should be understood that every member of a genus is, individually, an aspect of the invention. Also, aspects described as a genus or selecting a member of a genus should be understood to embrace combinations of two or more members of the genus. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

Features of the compositions and methods are described below. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. It will be understood that any feature of the methods or compounds described herein can be deleted, combined with, or substituted for, in whole or part, any other feature described herein.

### Device

The device of the present invention can be shown by FIG. 1. FIG. 1 shows an applicator device (10) comprising a cap (11), a body (12), and a rotating portion (13). The body (12) can also comprise a first indicator symbol (14). The rotating portion (13) can also comprise a second indicator symbol.

FIG. 2 shows a cross-sectional view taken along a longitudinal axis of the device (10). The device can also comprise a reservoir (20), a rotating member (21), a rotating dial (22), and a platform (23) forming a bottom portion of the reservoir (20). The device can also comprise an applicator (25).

The device (10) can be substantially cylindrical, rectangular, or any other shape suitable for application of an oral care composition to a surface of the oral cavity.

The cap (11) can be a screw cap, an over cap, or any other suitable cap for enclosing the applicator (25) when not in use. The cap (11) can be modified to allow the device (10) to rest on the cap (11) in a vertical position on a suitable surface, such as a countertop.

The body (12) can comprise the reservoir (20), rotating member (21), rotating dial (22), and the platform (23) forming the bottom portion of the reservoir (20). The body (12) can remain stationary when a user rotates the rotating portion (13).

The body (12) can be sized for use in self-application or sized for application by dental professional. The body (12) can be straight or substantially straight. The body (12) can also be curved, angled, or bendable to facilitate application to hard to reach oral care surfaces, such as tooth surfaces in the back of the oral cavity.

The body (12) can be made from any suitable material, such as for example, a polymer, a polymer alloy, an elastomer, a metal, a metal alloy, glass, and/or combinations thereof. The body (12) can have gripping elements or aesthetic elements. The body (12) can be opaque, translucent, transparent, and/or combinations thereof. As described herein, the applicator (25) can be designed to facilitate transfer of an oral care composition from the applicator (25) to an oral cavity surface.

The body (12) can also comprise a ratchet wheel with a top, bottom, and a plurality of teeth at the top of the ratchet wheel. There can also be a securing seat above the ratchet wheel, the securing seat can comprise a top, bottom, and a plurality of teeth opposite to the plurality of teeth of the ratchet wheel.

The applicator (25) can be made from a material that is suitable for application to oral care surfaces. For example, the applicator (24 can be made from food & drug grade materials, materials on the GRAS (Generally Regarded As Safe) list, or other applicable materials approved for use within the oral cavity according to local laws.

The applicator (25) can comprise a tip, one or more bristles, a rollerball, a swab, a needle, or any other suitable applicator for applying a composition to the oral cavity, such as the teeth and/or gums. The applicator tip can be made from any suitable material, such as for example, a polymer, a polymer alloy, an elastomer, a metal, a metal alloy, glass, bristles, and/or combinations thereof. The applicator can be free of bristles.

Suitable polymer materials for the applicator tip include, but are not limited to, nylon, polypropylene, polyethylene, polyethylene terephthalate, and/or combinations thereof.

Suitable elastomer materials include, but are not limited to, thermoplastic elastomers, a styrenic, a copolyester, a polyurethane, a polyamide, a polyolefin blend, a polyolefin alloy, a reactor TPO, a polyolefin plastomer, a polyolefin elastomer, and/or combinations thereof. Suitable elastomers include, for example, an elastomer made from PolyOne® under the Versaflex™ product lines or from Hapco, Inc under the Steralloy™ product line.

The applicator (25) can include one or more orifices to allow the oral care composition stored within the reservoir (20) to be extruded into or out of the applicator (25) to be applied to the oral cavity. The one or more orifices of the applicator (25) can be in fluid communication with the reservoir (20).

The reservoir (20) can be the void created by the inner surface of the body (12), the upper surface of the platform (23), and the applicator (25). The reservoir (20) can comprise the oral care composition until it is dispensed to the applicator (25) and ultimately applied to one or more surfaces within the oral cavity.

The reservoir (20) can comprise from about 0.1 g to about 100 g, from about 0.5 g to about 10 g, from about 1 g to about 5 g, or from about 0.01 g to about 10 g of the oral care composition.

The device (10) can be a twist-up pen-type dispenser, as described in U.S. Pat. No. 7,201,527, the apparatus as described in U.S. Pat. No. 8,602,774, or the applicator as described in U.S. Patent Application Publication No. 2006/0275225, all of which are herein incorporated by reference.

### Oral Care Composition

The oral care composition that can be dispensed from the reservoir (20) and onto/into the applicator (25) can be any suitable liquid, semiliquid, or any other extrudable composition comprising one or more oral care active agents. For example, the oral care composition can be a whitening composition and include a whitening agent. The oral care composition can include fluoride and/or tin.

The oral care composition may include an effective amount of an anti-caries agent. The oral care composition can comprise a fluoride ion source.

The fluoride ion source may be present in an amount sufficient to give a suitable fluoride ion concentration in the composition according to local laws and regulations, for example the anti-caries monograph at the FDA. The oral care composition can comprise from about 0.0025% to about 20%, from about 0.0025% to about 10%, from about 0.01% to about 5%, or from about 0.0025% to about 2%, by weight of the oral care composition, of the fluoride ion source.

The fluoride ion source can comprise stannous fluoride, sodium fluoride, potassium fluoride, amine fluoride, sodium monofluorophosphate, zinc fluoride, and/or combinations thereof.

The fluoride ion source and the metal ion source can be the same compound, such as for example, stannous fluoride, which can generate tin ions and fluoride ions. Additionally, the fluoride ion source and the tin ion source can be separate compounds, such as when the metal ion source is stannous chloride and the fluoride ion source is sodium monofluorophosphate or sodium fluoride.

The oral care composition can comprise a metal ion source. Suitable metal ion sources include stannous ion sources, zinc ion sources, copper ion sources, silver ion sources, magnesium ion sources, iron ion sources, sodium ion sources, and manganese (Mn) ion sources, and/or combinations thereof. The metal ion source can be a soluble or a sparingly soluble compound of stannous, zinc, or copper with inorganic or organic counter ions. Examples include the fluoride, chloride, chlorofluoride, acetate, hexafluorozirconate, sulfate, tartrate, gluconate, citrate, malate, glycinate, pyrophosphate, metaphosphate, oxalate, phosphate, carbonate salts and oxides of stannous, zinc, and copper.

Stannous, zinc and copper ions are derived from the metal ion source(s) can be found in the multi-phase oral care composition an effective amount to provide an oral care benefit or other benefits. Stannous, zinc and copper ions have been found to help in the reduction of gingivitis, plaque, sensitivity, and improved breath benefits. An effective amount is defined as from at least about 500 ppm to about 20,000 ppm metal ion of the total composition, preferably from about 2,000 ppm to about 15,000 ppm. More preferably, metal ions are present in an amount from about 3,000 ppm to about 13,000 ppm and even more preferably from about 5,000 ppm to about 10,000 ppm. This is the total amount of metal ions (stannous, zinc, copper and mixtures thereof) that is present in the compositions for delivery to the tooth surface.

Other metal ion sources can include minerals and/or calcium containing compounds, which can lead to remineralization, such as, for example, sodium iodide, potassium iodide, calcium chloride, calcium lactate, calcium phosphate, hydroxyapatite, fluoroapatite, amorphous calcium phosphate, crystalline calcium phosphate, sodium bicarbonate, sodium carbonate, calcium carbonate, oxalic acid, dipotassium oxalate, monosodium monopotassium oxalate, casein phosphopeptides, and/or casein phosphopeptide coated hydroxy apatite.

The metal ion source may comprise a metal salt suitable for generating metal ions in the oral cavity. Suitable metal salts include salts of potassium (K), silver (Ag), magnesium (Mg), iron (Fe), sodium (Na), and manganese (Mn) salts, or combinations thereof. Preferred salts include, without limitation, gluconates, chlorates, citrates, chlorides, fluorides, and nitrates, or combinations thereof.

The oral care composition can comprise at least about 0.005%, from about 0.005% to about 10%, from about 0.01% to about 5%, from about 0.01% to about 2%, or from about 0.1% to about 1% of a metal ion source by weight of the oral care composition.

Tin ions, such as stannous ions, are used in oral care compositions to deliver benefits such as, for example, enamel care and cavity protection. Suitable tin ion sources include stannous chloride, stannous fluoride, stannous bromide, stannous iodide, stannous acetate, stannous gluconate, stannous oxalate, stannous sulfate, stannous lactate, stannous tartrate stannous carbonate, stannic chloride, stannic fluoride, stannic iodide, stannous citrate, stannic nitrate, stannous peptides, stannous proteins, and stannous phosphate, and combinations thereof. Preferably, the ion source is stannous fluoride, stannous chloride, and/or combinations thereof.

The oral care compositions of the present invention may comprise a tin ion source in the amount ranging from about 0.01% to about 5%, from about 0.05% to about 4%, from about 0.01% to about 10%, or from about 0.075% to about 3%.

The oral care composition can also comprise a whitening composition, the whitening composition comprising a whitening agent. The oral care composition may comprise from about 0.1% to about 10%, from about 0.2% to about 5%, from about 1% to about 5%, or from about 1% to about 40%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, and combinations thereof. Suitable peroxides include hydrogen peroxide, solid peroxides, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

The oral care composition can also include those compositions described in in U.S. Patent App. Pub. No. 2018/0133119 and U.S. Patent App. Pub. No. 2018/0133121, which are herein incorporated by reference.

The oral care composition can also include various thickening materials to thicken the composition. The oral care compositions herein may include one or more thickening agents. A thickening agent may be used in an amount from about 0.01% to about 15%, or from about 0.1% to about 10%, or from about 0.1% to about 5%, by weight of the oral care composition. Non-limiting examples may include those described in US 2008/0081023 A1 at paragraphs 134 to 137, and the references cited therein.

The oral care composition can comprise a linear sulfated polysaccharide as a thickening agent. Carageenans or carageenans are one example of a linear sulfated polysaccharide. Generally, carageenans can vary based upon the degree of sulfation that includes: Kappa-carrageenan, Iota-carrageenan, and Lambda-carrageenan. Combinations of carageenans can be used. The oral care composition can contain from about 0.1% to about 3%, of a linear sulfated polysaccharides by weight of the oral care composition, from about 0.5% to about 2%, from about 0.6% to about 1.8%, or combinations thereof.

The oral care composition can comprise a silica agent, preferably a thickening silica obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167). The oral care composition can include from about 0.5% to about 5% by weight of the oral care composition of a silica agent, preferably from about 1% to about 4%, alternatively from about 1.5% to about 3.5%, alternatively from about 2% to about 3%, alternatively from about 2% to about 5% alternatively from about 1% to 3%, alternatively combinations thereof.

The thickening agent can comprise a carboxymethyl cellulose (“CMC”). CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9 M3SF Aqualon™ TM9A; Aqualon™ TM12A). The thickening agent can contain from about 0.1% to about 3% of a CMC by weight of the oral care composition, preferably from about 0.5% to about 2%, alternatively from about 0.6% to about 1.8%, alternatively combinations thereof.

### Dispensing the Oral Care Composition

The oral care composition can be dispensed out of the device (10) by having a user rotate the rotating portion (13).

Upon rotation of the rotation of the rotating portion (13), the rotating dial (22) can be rotated, which can cause the rotating member (21) to move towards the applicator (25). The rotation of the rotating portion (13) can also cause a ratchet wheel with a top, bottom, and a plurality of teeth at the top to rotate. Each time a tooth of the ratchet wheel rotates, an audible click can be heard, as the plurality of teeth of the ratchet wheel interact with the plurality of teeth of the securing seat. The rotating member (21) can be threaded with screw threads for mounting to the interior surface of the body (12), as shown in FIG. 2. The platform (23) can be associated with and/or attached to the rotating member (21). When the rotating dial (22) is rotated in one direction, the rotating member (21) can rotate in the same direction within the screw threads, as shown in FIG. 4. The movement of the rotating member (21) can move the platform (23) to squeeze the oral care composition towards the one or more orifices of the applicator (25). This process is well known in the art and further described in detail in U.S. Pat. No. 7,201,527, which is herein incorporated by reference in its entirety.

### Indicator Symbols

When a user rotates the rotating portion (13), it can be challenging for the user to quantify the amount of material squeezed from the reservoir (20) and onto/into the applicator (25). Thus, the present invention is directed towards dosing indicator symbols to denote the distance the user has rotated the rotating portion (13).

In one example, as shown in FIG. 1, the body (12) can comprise a first dosing indicator symbol (14) and the rotating portion (13) can include a second dosing indicator symbol (15). FIG. 1 displays the device when the first (14) and second (15) dosing indicator symbols are aligned. FIG. 3 displays the device when the second dosing indicator symbol (15) has been rotated in one direction with the rotating portion (13) of the device (10). As shown in FIG. 4, the rotation of the rotating portion (13) pushing the platform (23) towards the applicator (25) and expels a portion of the composition from the reservoir (20).

A defined distance of the movement of the second dosing indicator (15) can correspond to an amount of composition dispensed from the device (10). For example, a user can be instructed to turn the rotating portion (13) a full turn, thereby starting and stopping the rotation when the first (14) and second (15) dosing indicator symbols are aligned. Alternatively, the rotating portion (13) can include a scale of amounts, such as in volume, mass, or weight of material, that can be paired with the first dosing indicator symbol (14) on the body (12) of the device (10), to indicate the amount of composition dispensed with a particular distance rotated.

The dosing indicator symbol can be any visual and/or aesthetic feature that can allow the user to determine how far the rotating portion (13) has been rotated. The dosing indicator symbol(s) can be a printed symbol, color, shape, graphic, picture, word, logo, etc. on the packaging sleeve or on the device (10) itself or the dosing indicator symbols can be a raised or depressed feature of the device (10), such as embossing, debossing, ridge, or the like. Suitable examples of dosing indicator symbols can include, but are not limited to, arrows, dots, circles, rectangles, triangles, lines, words, such as “START” and/or “STOP”, circles, ovals, letters, figures, or the like. Dosing indicator symbols can complete a graphic when aligned properly. For example, a portion of a graphic can be on the body (12) and the remaining portion of a graphic can be on the rotating portion (13). When aligned, the graphic is visually complete.

The dosing indicator symbol(s) can also differ in transparency from the body (12) and/or rotating portion (13). For example, if the body (12) is transparent or translucent, the dosing indicator symbol(s) can be opaque.

The first (14) and second (15) dosing indicator symbols can be identical, substantially similar, or different in appearance. The first dosing indicator symbol (14) can be a symbol, as described herein, while the second dosing indicator symbol (15) can be a scale based on mass, volume, and/or weight that spans the at least a portion of the outer surface of the rotating portion (13). The first (14) and second (15) dosing indicator symbols can be in direct contact when aligned, can have a gap between the first (14) and second (15) dosing indicator symbols when aligned, and/or the first (14) and second (15) dosing indicator symbols can be substantially visually aligned without direct contact.

A full turn of the rotating portion (13) can be when the first (14) and second (15) dosing indicator symbols start aligned and are rotated in one direction until the first (14) and second (15) dosing indicator symbols become aligned again. A full turn of the rotating portion (13) can correspond to the dispensing of from about 0.01 g to about 1 g, from about 0.05 g to about 0.25 g, or about 0.1 g of the oral care composition.

In the example where the oral care composition is a whitening composition and the whitening composition comprises hydrogen peroxide, a full or partial turn of the device (10) can correspond to the correct dosage of hydrogen peroxide that should be applied to the oral cavity in a single application to effectively whiten teeth without experiencing irritation.

In the example where the oral care composition comprises fluoride, a full or partial turn of the device (10) can correspond to the correct dosage fluoride that should be applied to the oral cavity in a single application according to the relevant regulatory agency, such as the United Stated Food and Drug Administration (FDA).

### Methods

Also disclosed herein is a method for dispensing and/or applying an oral care composition using the dosing indicator symbols and illustrated in FIG. 1-4.

A user can dispense a defined amount of an oral care composition by using the dosing indicator symbols as shown in FIG. 1-4. First, a user can align the first (14) and second (15) dosing indicator symbols as shown in FIG. 1. Next, a user can rotate the rotating portion (13) of the device (10) in one direction as shown in FIG. 3. As described herein, the rotation of the rotating portion (13) dispenses the oral care composition stored within the reservoir (20) from the device (10) and into/onto the applicator (25). The user can stop rotating the rotation portion (13) once the first (14) and second (15) dosing indicator symbols are aligned. Finally, the oral care composition can then be applied to an oral cavity surface, such as the teeth in the example of fluoride or peroxide or the teeth and/or the gums in the case of tin.

Additionally, or alternatively, the second dosing indicator symbol (15) can be a scale of volume, weights, or mass. In this case, the rotation of the rotation portion (13) can instead be instead stopped when the first dosing indicator symbol (14) is aligned with the desired value, which may or may not be exactly a full rotation as shown in FIG. 1 and FIG. 3.

### Examples

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

A whitening pen was provided by WSD Labs and the reservoir of the whitening pen was loaded with the whitening composition of TABLE 1.

TABLE 1
Whitening Composition

| | |
|---|---|
| Hydrogen Peroxide (35%) | 8.700 |
| Glycerin, USP | 20.500 |
| Water | 65.400 |
| Sodium Acid Pyrophosphate | 1.000 |
| Carbopol ® 956 Polymer³ (CAS# is 134499-38-0) | 2.000 |
| Sodium Hydroxide (50% solution) | 0.900 |
| Flavor | 1.000 |
| Sucralose, USP | 0.500 |

³Available from the Goodrich Corporation (Akron, Ohio, USA)

It was observed that a full rotation of the rotating portion was 24 “clicks” or 24 movements of the internal ratchet wheel within the body of the whitening pen. Thus, an experiment was conducted to measure the amount of material that was dispensed from the pen after a full rotation of the rotating portion. The whitening pen was weighed. Then, the rotating portion was rotated by a defined number of “clicks” to dispense an amount of the whitening composition. The whitening composition was then removed from the applicator tip in a manner similar to how a user would apply a composition to an oral cavity. The whitening pen was then re-weighed. The displaced weight was equivalent to the amount of material dispensed.

The results of the dosing check are described in TABLE 2. In the first trial, after 25 “clicks,” only 0.04 g of material was dispensed onto the oral cavity. This was due to the fact that the headspace above the reservoir of the whitening composition was empty prior to the first use. Thus, less material was dispensed with the first full rotation.

After the initial priming of the whitening pen, subsequent rotations led to 0.1 g, 0.1 g, 0.12 g, and 0.11 g of dispensed material based on the # of clicks provided in TABLE 2. If trial 1 is excluded, approximately 0.1 g of material was applied to the oral cavity. Additionally, demarcations were added on the device to calculate the # of clicks that represented a full rotation. This resulted in the observation that in the whitening pen utilized, a full rotation was represented with 24 “clicks.” As such, using the averaged dispensed material per full rotation was calculated by Formula I.

TABLE 2
Dispensed Composition

| | | | Dispensed Material | # of |
|---|---|---|---|---|
| Trial | Initial weight of Pen (g) | Weight after a full rotation of Pen (g) | (g) | “Clicks” |
| 1 | 15.4 | 15.36 | 0.04 | 25 |
| 2 | 15.33 | 15.23 | 0.1 | 30 |
| 3 | 15.18 | 15.08 | 0.1 | 25 |
| 4 | 15.04 | 14.92 | 0.12 | 30 |
| 5 | 14.89 | 14.78 | 0.11 | 25 |

$Average Dispensed Material per full rotation = \frac{\sum Dispensed Material}{\sum \# of Clicks} * 24 \qquad$ Formula I

The total dispensed material was divided by the total # of clicks shown in TABLE 2. This resulted in 0.0039 g per “click.” As the # of “clicks” per full rotation was determined be 24, this number was multiplied by 24 to obtain the amount dispensed per full rotation of the rotating portion. The average dispensed material per full rotation was determined to be 0.094 g.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A whitening pen comprising:
(a) a whitening composition;
(b) a body;
(c) a rotating portion;
(d) a dosing indicator, the dosing indicator comprising:
(i) a first dosing indicator symbol on the body, and
(ii) a second dosing indicator symbol on the rotating portion.

2. The whitening pen of claim 1, wherein a full rotation of the rotating portion is indicated by the first dosing indicator symbol and the second dosing indicator symbol start aligned, and after the rotation of the rotating portion in a single direction, the first dosing indicator symbol and second dosing indicator symbol end aligned.

3. The whitening pen of claim 1, wherein a full rotation of the rotating portion releases from about 0.05 g to about 0.25 g of the whitening composition.

4. The whitening pen of claim 1, wherein the whitening composition comprises a peroxide.

5. The whitening pen of claim 4, wherein the peroxide comprises hydrogen peroxide, carbamide peroxide, or combinations thereof.

6. The whitening pen of claim 1, wherein the whitening pen comprises an applicator.

7. The whitening pen of claim 1, wherein the first dosing indicator symbol comprises an arrow, a line, a circle, an oval, a letter, a word, a picture, a graphic, or combinations thereof.

8. The whitening pen of claim 7, wherein the second indicator dosing symbol comprises an arrow, a line, a circle, an oval, a letter, a word, a picture, a graphic, or combinations thereof.

9. The whitening pen of claim 8, wherein the first and second dosing indicator symbols are identical.

10. The device of claim 7, wherein the second dosing indicator symbol comprises a volume scale to denote the volume of material dispensed per distance rotated.

11. The whitening pen of claim 6, wherein the body comprises a reservoir, the reservoir comprising the whitening composition.

12. The whitening pen of claim 1, first dosing indicator symbol and the second indicator symbol are portions of a graphic, figure, word, letter, number, symbol, or combinations thereof that, when aligned, display the entire graphic, figure, word, letter, number, symbol, or combinations thereof.

13. The whitening pen of claim 11, wherein the applicator comprises one or more orifices in fluid communication with the reservoir.

14. The whitening pen of claim 5, wherein about 0.1 g of the whitening composition is dispensed with one full rotation of the rotation portion.

15. A dispensing device comprising:
(a) an oral care composition;
(b) a body;
(c) a rotating portion;
(d) a dosing indicator, the dosing indicator comprising:
(i) a first dosing indicator symbol on the body, and
(ii) a second dosing indicator symbol on the rotating portion.

16. The device of claim 15, wherein the oral care composition comprises fluoride, peroxide, a metal ion, or combinations thereof.

17. The device of claim 16, wherein the fluoride comprises stannous fluoride, sodium fluoride, amine fluoride, sodium monofluorophosphate, or combinations thereof.

18. The device of claim 16, wherein the metal ion comprises stannous, zinc, copper, or combinations thereof.

19. The device of claim 16, wherein the peroxide comprises hydrogen peroxide or carbamide peroxide.

20. The device of claim 15, wherein the device comprises an applicator.

21. The device of claim 20, wherein the body comprises a reservoir, the reservoir comprising the oral care composition.

22. The device of claim 21, wherein the applicator comprises one or more orifices in fluid communication with the reservoir.

23. The device of claim 15, wherein the first dosing indicator symbol comprises an arrow, a line, a circle, an oval, a letter, a word, a picture, a graphic, or combinations thereof.

24. The device of claim 23, wherein the second dosing indicator symbol comprises an arrow, a line, a circle, an oval, a letter, a word, a picture, a graphic, or combinations thereof.

25. The device of claim 24, wherein the first and second dosing indicator symbols are identical.

26. The device of claim 24, wherein the second dosing indicator symbol comprises a volume scale to denote the volume of material dispensed per distance rotated.

27. The device of claim 15, wherein the device is free of bristles.

28. The device of claim 15, wherein a full rotation of the rotating portion is indicated by the first dosing indicator symbol and the second dosing indicator symbol start aligned, and after the rotation of the rotating portion in a single direction, the first dosing indicator symbol and second dosing indicator symbol end aligned.

29. The device of claim 15, wherein a full rotation of the rotating portion releases from about 0.05 g to about 0.25 g of the whitening composition.

30. A dispensing device comprising:
(a) an oral care composition, the oral care composition comprising fluoride or peroxide;
(b) a body;
(c) a rotating portion;
(d) a dosing indicator, the dosing indicator comprising:
(i) a first dosing indicator symbol on the body, and
(ii) a second dosing indicator symbol on the rotating portion.

31. A method of dispensing an oral care composition from a dispensing device comprising:
(a) providing a dispensing device, the dispensing device comprising:
(i) the oral care composition comprising fluoride or peroxide;
(ii) a body;
(iii) a rotating portion;
(iv) a dosing indicator comprising:
(1) a first dosing indicator symbol on the body, and
(2) a second dosing indicator symbol on the rotating portion;
(b) aligning the first dosing indicator symbol with the second dosing indicator symbol;
(c) rotating the rotating portion in one direction; and
(d) re-aligning the first dosing indicator symbol with the second dosing indicator symbol.
